SEQUENCE LISTING (14 sequences)

SEQ ID NO: 1 - protein, 6 aa, Artificial Sequence
  B1 peptide
INHHLG

SEQ ID NO: 2 - protein, 6 aa, Artificial Sequence
  B2 peptide
ISHHLG

SEQ ID NO: 3 - protein, 6 aa, Artificial Sequence
  B3 peptide
INHNLG

SEQ ID NO: 4 - protein, 6 aa, Artificial Sequence
  B4 peptide
ISHNLG

SEQ ID NO: 5 - protein, 6 aa, Artificial Sequence
  B5 peptide
FNHHLG

SEQ ID NO: 6 - protein, 6 aa, Artificial Sequence
  B6 peptide
FSHHLG

SEQ ID NO: 7 - protein, 6 aa, Artificial Sequence
  B7 peptide
FNHNLG

SEQ ID NO: 8 - protein, 6 aa, Artificial Sequence
  B8 peptide
FSHNLG

SEQ ID NO: 9 - protein, 5 aa, Artificial Sequence
  C2 peptide
FSHHL

SEQ ID NO: 10 - protein, 5 aa, Artificial Sequence
  C3 peptide
SHHLG

SEQ ID NO: 11 - protein, 4 aa, Artificial Sequence
  C4 peptide
HHLG

SEQ ID NO: 12 - protein, 3 aa, Artificial Sequence
  C5 peptide
HLG

SEQ ID NO: 13 - protein, 2 aa, Artificial Sequence
  C6 peptide
LG

SEQ ID NO: 14 - protein, 1 aa, Artificial Sequence
  C7 peptide
G

(19) United States
(12) Patent Application Publication
(10) Pub. No.: US 2019/0382445 A1
(43) Pub. Date: Dec. 19, 2019

(54) PEPTIDE HAVING MELANOGENESIS-INHIBITING ACTIVITY AND COMPOSITION COMPRISING SAME

(71) Applicant: RUBY CROWN CO. LTD., Daegu (KR)

(72) Inventors: Yong Chool BOO, Daegu (KR); Young Mi KIM, Daegu (KR)

(73) Assignee: RUBY CROWN CO. LTD., Daegu (KR)

(21) Appl. No.: 16/308,410

(22) PCT Filed: Jun. 9, 2017
(86) PCT No.: PCT/KR2017/006011

§ 371 Date: Dec. 7, 2018

(30) Foreign Application Priority Data
Jun. 9, 2016 (KR) 10-2016-0071515

(51) Int. Cl. C07K 7/06

(52) U.S. Cl. CPC C07K 7/06; A61K 38/00

(57) ABSTRACT

The present invention relates to a peptide having inhibitory activity against melanogenesis and a composition comprising the same and, more particularly, to a peptide having melanogenesis-inhibiting activity and a pharmaceutical, a cosmetic, and a food composition comprising the same as an effective ingredient for whitening the skin, the peptide comprising an amino acid sequence represented by General Formula (I) amino terminus-X1-X2-X3-Leu-X4-carboxy terminus, wherein X1 is Asn or Ser; X2 is an amino acid selected from the group consisting of Asn, His, and Asp; X3 is His or Asn; and X4 is Gly or Phe. Exhibiting the effect of very effectively inhibiting the activity of enzymes and proteins which are associated with melanin biosynthesis, without decreasing cell viability, the peptide according to the present invention can be usefully utilized in developing skin-whitening medications, cosmetics, and functional foods.

### TECHNICAL FIELD

The present invention relates to a peptide having melanin generation (‘melanogenesis’)-inhibiting activity and a composition including the same, more particularly, a peptide having melanogenesis-inhibiting activity, which includes an amino acid sequence represented by general formula (I) of: amino terminal-X1-X2-X3-Leu-X4-carboxy terminal, wherein X1 is Asn or Ser; X2 is any one amino acid selected from a group consisting of Asn, His and Asp; X3 is His or Asn; and X4 is Gly or Phe, as well as a whitening, pharmaceutical, cosmetic and/or food composition including the above peptide as an active ingredient.

### BACKGROUND ART

Melanin is a brown or black pigment that exists in the skin, hair, eyes, and some tissues wherein other pigments are deposited, and is very important to appearance of a person and also in maintaining skin homeostasis. Melanin biosynthesis in the skin is affected by a number of factors including changes in hormonal and nutritional states and, if melanin is not normally generated and melanin biosynthesis is disturbed, defective pigmentation classified as reduced pigmentation or excessive pigmentation may occur. Such defective pigmentation may be a genetic or acquired defect, may be temporary or permanent and may occur in some parts of or throughout the body. When melanin is abnormally accumulated or distributed, apparent signs such as freckles, dark spots and/or age-related spots, which are unlikable in appearance, may be caused. Therefore, preventing undesirable accumulation of melanin is of urgent interest in the cosmetics industry.

Melanocytes (melanin-forming cells) interact with external factors such as UV, drugs, etc. as well as the endocrine system in the body. A single melanocyte in the skin is surrounded by about 30 to 40 keratinocytes, and melanogenesis is controlled by a closed paracrine system. Melanin is synthesized from L-tyrosine by enzyme chain reaction in melanosomes, a cellular organelle within the melanocyte. Keratinocytes secrete a signal transduction material that stimulates melanocytes to mature melanosome and promotes melanin biosynthesis. Melanin in the mature melanosome is discharged from a dendrite of the melanocyte and delivered to cytoplasm of adjacent keratinocytes.

α-melanocyte stimulating hormone (α-MSH) is critical to generation of melanin. α-MSH consists of an amino acid sequence of Ac-Ser-Tyr-Ser-Met-Glu-His-Phe-Arg-Trp-Gly-Lys-Pro-Val-NH₂, and is an agent of melanocortin 1 receptor (MC1-R). If the agent is coupled to MC1-R, the MC1-R activates adenylate cyclase, which in turn increases cAMP and activates PKA. The PKA leads phosphorylation of a cAMP-responsive element binding protein transcription factor, resulting in activation of microphthalmia-associated transcription factor (MITF). MITF is also activated by Wnt, GSK3β and the MAPK signal transduction system and responds to various stimuli to thus control expression levels of enzymes involved in melanin biosynthesis such as tyrosinase (TYR), tyrosinase-related protein 1 (TYRP1), dopachrome tautomerase (DCT; also known as tyrosinase-related protein 2 (TRP2)), etc. Agouti signal transduction protein (Agouti) is known as an antagonist of MC1-R which competes with α-MSH and inhibits melanogenesis.

Peptides have attracted attention as an active ingredient in cosmetics and medical cosmetics (cosmoceuticals) because of bioactivity suitable for skin care, and diverse peptides have already been used as cosmetic ingredients. Amino acid sequences of peptides are highly variable and may serve a variety of functions. Moreover, peptides are degraded into natural amino acids without toxicity. However, peptides have drawbacks of high peptide synthesis cost and inefficient skin penetration due to ionic property. Palmitoyl pentapeptide-4 (KTTKS), glycyl-histidyl-lysine (GHK)-Cu and acetyl hexapeptide, (Argireline), etc. can alleviate a specific aspect of skin aging. As peptides with melanogenesis inhibitory effects, disulfanyl peptide and Angio-3 (SFKLRY-NH₂) have been reported.

In order to develop a peptide inhibitor to suppress cellular melanogenesis, the present inventors have applied positional scanning of a synthetic peptide combinatorial library (PS-SPCL) which has been effectively used for screening synthetic peptide. Using murine melanoma B16-F10 cell lines stimulated with PS-SPCL and α-MSH has determined peptides expected to have melanogenes is inhibiting activity, and then, identified effects thereof.

### DISCLOSURE

### Technical Problem

The present inventors screened for peptides consisting of 5 to 6 amino acids based on positional scanning of a synthetic peptide combinatorial library (PS-SPCL) and identified a novel peptide having intercellular melanogenesis inhibiting activity, thereby completing the present invention.

Therefore, an object of the present invention is to provide a peptide having melanogenesis inhibiting activity, which has an amino acid sequence represented by general formula (I) below.

(I) an amino terminal-X1-X2-X3-Leu-X4-carboxy terminal;

wherein,

X1 is Asn or Ser;

X2 is any one amino acid selected from a group consisting of Asn, His and Asp;

X3 is His or Asn; and

X4 is Gly or Phe.

Another object of the present invention is to provide a pharmaceutical composition with whitening effects, which includes the peptide described above as an active ingredient.

Another object of the present invention is to provide a cosmetic composition with whitening effects, which includes the peptide described above as an active ingredient.

A further object of the present invention is to provide a food composition with whitening effects, which includes the peptide described above as an active ingredient.

### Technical Solution

In order to accomplish the above objects, the present invention provides,

a peptide having melanogenesis inhibiting activity, which has an amino acid sequence represented by general formula (I) below.

(I) an amino terminal-X1-X2-X3-Leu-X4-carboxy terminal;

wherein,

X1 is Asn or Ser;

X2 is any one amino acid selected from a group consisting of Asn, His and Asp;

X3 is His or Asn; and

X4 is Gly or Phe.

In order to accomplish another object of the present invention,

there is provided a pharmaceutical composition with whitening effects, which includes the peptide described above as an active ingredient.

In order to accomplish another object of the present invention,

there is provided a cosmetic composition with whitening effects, which includes the peptide described above as an active ingredient.

In order to accomplish a further object of the present invention,

there is provided a food composition with whitening effects, which includes the peptide described above as an active ingredient.

### Advantageous Effects

The peptide according to the present invention is very effective at inhibiting activity of the enzyme and protein in relation to melanogenesis while not deteriorating cell viability.

### DESCRIPTION OF DRAWINGS

FIG. 1 is a scanning position (PS-SPCL) result in a synthetic peptide combinatorial library for melanogenesis in cells. Rodent melanoma cell lines, B16-F10 cells, were stimulated with a vehicle control group or the corresponding peptide pool, and a content of melanin was measured. Each panel represents the results obtained from the hexapeptide pool, and amino acid sequences of X and O at the top of the panel denote positional characteristics of the amino acids in the hexapeptide pool. O position is determined as one of 19 types of L-amino acids excluding cysteine, respectively, while the remaining five X positions are composed of a mixture of the other 19 types of L-amino acids.

FIG. 2 is an experimental result showing the effect of each hexapeptide on cellular melanin biosynthesis. FIG. 2A represents a peptide wherein the type of amino acids fixed at 3 positions in the amino acid sequence of the amino acids, while the type of amino acids is altered at the other 3 positions. FIG. 2B represents a peptide wherein the amino acid sequence was defined. The cell-based assay in FIG. 2 is conducted by treating B16-F10 cells with a vehicle or peptide at a predetermined concentration, stimulating the same with α-MSH at 100 nM, and measuring absorbance at 475 nm after 72 hours to determine melanin content. Data are expressed in percent (%) as mean ± standard error (mean±SE, n=3) compared to the vehicle control group. In the figures, # means p<0.05 compared to the control group and * means p<0.05 compared to cells stimulated only with α-MSH. The amino acid sequences described in FIG. 2 are all represented in the direction from the amino terminal to the carboxy terminal. An amino functional group (—NH₂) shown in the peptide terminal is one added by carboxy terminal amidation used in peptide synthesis.

FIG. 3 shows effects of the peptides wherein amino acid sequences were defined. The cell-based Assay in FIG. 3 is conducted by treating B16-F10 cells with a vehicle or peptide at a predetermined concentration, stimulating the same with 100 nMα-MSH0 nM, and measuring the absorbance at 475 nm after 72 hours to determine the melanin content. Data are expressed in percent (%) as mean±standard, error (mean±SE, n=3) compared to the vehicle control group. In the figures, # means p<0.05 compared to the control group and * means p<0.05 compared to cells stimulated only with α-MSH. The amino acid sequences described in FIG. 2 are all represented in the direction from the amino terminal to the carboxy terminal. An acetyl group (Ac—) shown in the peptide amino terminal or an amino functional group (—NH₂) shown in the carboxy terminal is one added by chemical synthesis of peptide.

FIG. 4 is an experimental result showing effects of B6 peptide on cell viability of B15-F10 cells, melanin content and protein levels of a melanin biosynthetic enzyme. FIG. 4A shows the cell viability identified by MMT assay after treatment of B6 peptide at different concentrations for 24 hours. FIG. 4B shows extracellular and intercellular melanin contents of the cell subjected to pre-treatment using B6 peptide at different concentrations for 60 minutes and then stimulation using 100 nM α-MSH 0 nM to B6 for 72 hours. The melanin content was normalized with respect to the total protein content. FIG. 40 shows the Western blot results using lysate of cells stimulated with 100 nM α-MSH 0 nM for 24 hours or 48 hours after pre-treatment using B6 peptide at different concentrations for 60 minutes. TYR, TYRP1 and DCT protein levels were normalized with respect to β-actin. Data are expressed in percent (%) as mean±standard error (mean±SE, n=3) compared to the vehicle control group. In the figures, # represents p<0.05 compared to the control group and * represents p<0.05 compared to cells stimulated only with α-MSH.

FIG. 5 is an experimental result showing effects of peptide B6 on cell viability of human epidermal melanocytes (HEM), melanin content and human tyrosinase (TYR) enzymatic activity. FIG. 5A shows the cell viability measured by MTT assay after pre-treatment using the peptide B6 at indicated concentrations for 24 hours. FIG. 5B shows the melanin content in the cells stimulated with 100 mM α-MSH at in the presence or absence of B6 peptide, while FIG. 5C shows the melanin content in the cells stimulated with 4.0 mM L-tyrosine for 6 days. The intracellular and extracellular melanin contents were normalized with respect to the total protein content. FIG. 5D shows effects of B6 peptide on enzymatic activity of human tyrosinase. Data are expressed in percent (%) as mean±standard error (mean±SE, n=3) compared to the vehicle control group. In the figures, #represents p<0.05 compared to the control group and * represents p<0.05 compared to cells stimulated only with tyrosine.

### BEST MODE

Hereinafter, the present invention will be described in more detail.

However, the following examples are illustrative only and duly not construed to limit the contents of the present invention.

<Experimental Method>

Synthesis of Peptide

A synthetic hexapeptide combinatorial library package was purchased from the Peptide Library Support Facility at the Pohang University of Science and Technology. The library consists of 6-positional sub-libraries, that is, OXXXXX, XOXXXX, XXOXXX, XXXOXX, XXXXOX and XXXXXOO positions, wherein any one of 19 types of L-amino acids is present at each O position, while X positions are composed of an equimolar mixture of the other L-amino acids except for cysteine. Each peptide in the library was synthesized through C-terminal amidation. The library consists of 114 tubes (6 types of positions×19 types of amino acids), and the total peptide concentration in each tube was 30 mM, the concentration of each peptide was 12 nM (30 mM/19⁵).

Further, a peptide pool and individual peptides were prepared by the peptide customization service of Peptron Co. (Daejeon, Korea). In Example 2, peptides chemically synthesized through carboxy terminal amidation were used. In Example 5, some peptides were chemically synthesized through amino terminal acetylation as well as carboxy terminal amidation and used. Types and purity of the peptides were determined by mass spectrometry (MS) and high performance liquid chromatography (HPLC).

Cell Culture

Cells were cultured in a humidified incubator at 37° C., 5% CO₂ conditions. Murine melanoma B16-F10 cell line was purchased from the American Type Culture Collection (Manassas, Va., USA) and cultured in Dulbecco's Modified Eagle Medium containing 10% FBS (fetal bovine serum) and antibiotics (100 U/mL) penicillin, 0.1 mg/mL streptomycin, 0.25 μg/mL amphotericin B). A human epidermal melanocyte (HEM) derived from the foreskin of a newborn infant was purchased from Cascade Biologics (Portland, Oreg., USA) and then cultured in Medium 254 containing human melanocyte growth supplements (Cascade Biologies) and antibiotics.

Cell Viability

Cell viability was determined by 3-(4,5-dimethylthiazol-2-yl)-2,5-diphenyltetrazolium bromide (MTT) assay. B16-F10 cell line and HEM were treated with test peptides at different concentrations for 24 hours. After washing with PBS, the cells were in a culture medium 100 including 1 mg/mL MTT (Amresco, Solon, Ohio, USA) added thereto for 3 hours. After removing the culture medium, formazan accumulated in the cells was accumulated using isopropanol and subjected to determination of absorbance at 595 nm using a SPECTROstar Nano microplate reader (BMG LABTECH GmbH, Ortenberg, Germany).

Melanogenesis-Inhibiting Activity

Melanogenesis-inhibitory effects of the analyte (the peptides) was confirmed using the B16-F10 cell line and HEM. B16-F10 cells were treated with a test peptide at different concentrations for 60 minutes and stimulated using a 100 nM α-MSH for 72 hours. While HEM was also treated with the test peptide at different concentrations for 60 minutes and cultured with 100 nM α-MSH or 4.0 mM L-tyrosine while replacing the culture medium every 48 hours for 6 days. Using the culture medium used for culturing cells (conditioned medium), extracellular melanin level was measured. On the other hand, intercellular melanin was extracted with 0.1M NaOH at 60° C. for 60 minutes. A melanin content was determined by measuring an absorbance at 475 nm according to a spectroscopic method. The resultant values were subjected to normalization with respect to the total protein content of the cell.

Western Blot

Protein lysis buffer (lysis buffer, pH 7.2) was composed of 10 mM Tris-HCl, 150 mM NaCl, 5 mM EDTA, 0.1% sodium dodecyl sulfate (SDS), 1% Triton X-100, 1% deoxycholate, 1 mM phenylmethylsulfonyl fluoride and protease inhibitor cocktail (Roche, Mannheim, Germany). The protein contained in Laemmli sample buffer was modified by heating at 95° C. for 5 minutes, subjected to electrophoresis in 10% SDS-PAGE, and then transferred to polyvinylidene difluoride membranes (Amersham Pharmacia, Little Chalfont, UK). The transfer membrane was reacted with a primary antibody at 4° C. overnight and then reacted with a secondary antibody, which was conjugated with a proper horse radish peroxidase (Cell Signaling, Danvers, Mass., USA), at room temperature for 1 hour. TYR, TYRP1, DCT, and b-actin were purchased from Santa Cruz Biotechnology (Santa Cruz, Calif., USA). Protein bands were detected using the Western Reagent kit picoEPD (ELPIS-Biotech, Daejeon, Korea), and a protein concentration assay was performed according to NIH image program.

### Tyrosinase Enzymatic Activity

Enzymatic activity of human tyrosinase (TYR) was carried out using a cell-free extract of human embryonic kidney 293 cells (HEK293-TYR), which permanently express human TYR. TYR enzymatic activity was measured with L-tyrosine as an enzyme substrate and 3,4-dihydroxyphenylalanine as a cofactor. The reaction mixture consisted of 100 mM sodium phosphate (pH 6.8), 0.5 mM L-tyrosine, 1 μM 3,4-dihydroxyphenylalanine, HEK293-TYR cell lysates (40 μg protein) and the test substance (peptide) at a specific concentration. The reaction mixture was placed in a 96-well plate and reacted at 37° C., followed by measurement of a change in absorbance at 475 nm using a SPECTROstar Nano microplate reader (BMG LABTECH GmbH Ortenberg, Germany). The relative enzymatic activity was calculated by Formula: (Compared to control %)=(C-D)/(A-B)×100; A and B are changes in absorbance of the control group over time in the presence or absence of TYR, respectively; C and D are changes in absorbance of the test group overtime in the presence or absence of TYR, respectively.

Statistical Analysis

Data show the results of at least three independent experiments as the mean±standard error (mean±SE). Statistical significance was determined by Student's t-test with respect to p<0.05.

### EXAMPLE 1

Identification of Peptides Having Melanogenesis-Inhibiting Activity using PS-SPCL

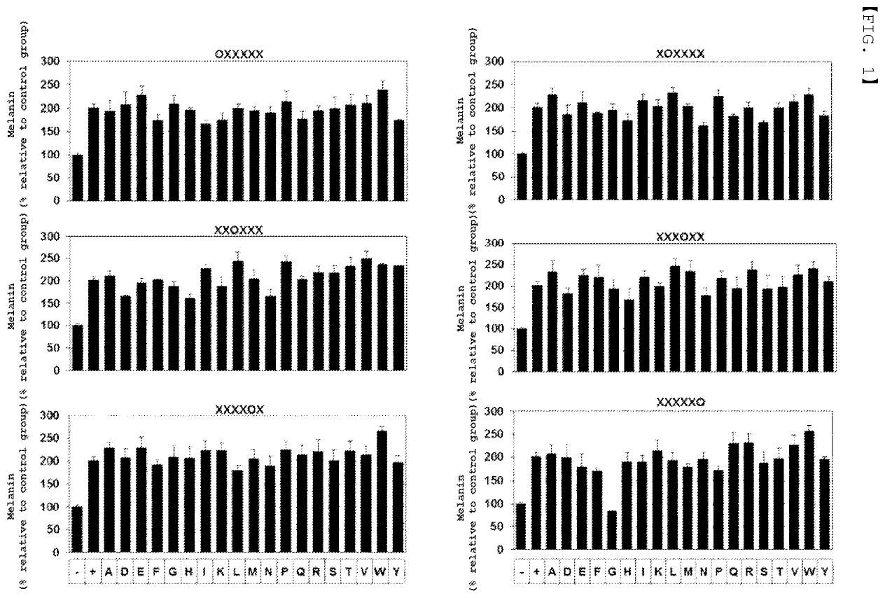

Screening using PS-SPCL was conducted by treating B16-F10 melanoma cells stimulated by α-MSH using all peptide pools in the synthesis hexapeptide combinatorial library, and then, observing effects of the stimulated cells on melanogenesis (FIG. 1).

As expected, α-MSH increased melanin content in B16-F10 cells, which was attenuated to different extents by various hexapeptide pools at 1.5 mM concentration. Thereamong, in particular, the hexapeptide pool having at least 25% inhibition on melanogenesis was identified. Depending on the known amino acid sequence of the peptide pool as well as the test result, a sequence of the peptide having melanogenesis activity was determined to include: amino acids Ile and Phe selected at the first position from the amino terminal; Asn and Ser selected at the second position; one of Asn, His and Asp selected at the third position; His and Asn selected at the fourth position; Leu selected at the fifth position; and Gly and Phe selected at the sixth position. That is, depending upon position scanning results, the amino acid sequence of the hexapeptide having melanogenesis-inhibiting activity was expected as follows: (Ile/Phe)-(Asn/Ser)-(Asn/His/Asp)-(His/Asn)-Leu-(Gly/Phe).

### EXAMPLE 2

Melanogenesis Inhibitory Effects of Hexapeptide Mixtures and Individual Peptides

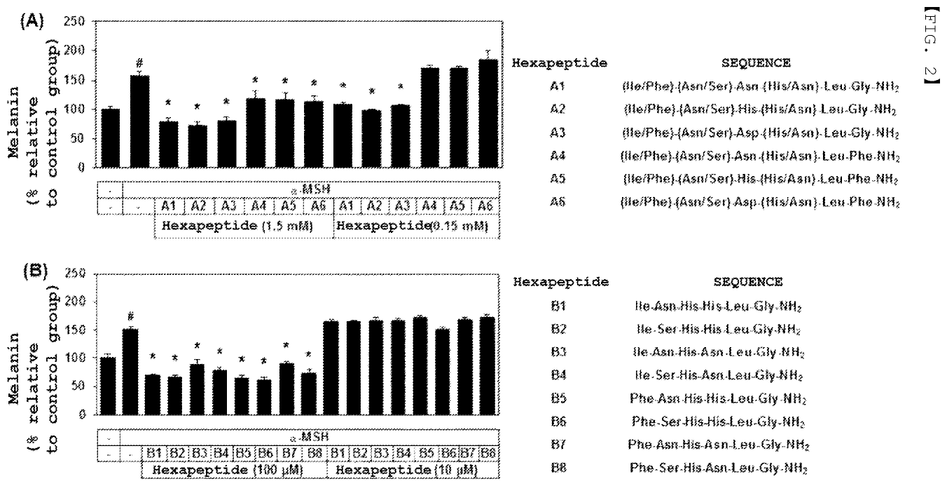

In accordance with the results of PS-SPCL in the foregoing example, 6 hexapeptide mixtures, each of which consists of 8 types of peptides, were synthesized and effects thereof on melanogenesis were demonstrated (FIG. 2). The hexapeptide mixture included: an equimolar mixture of Ile and Phe at the first position from the amino terminal; an equimolar mixture of Asn and Ser at the second position; an equimolar mixture of His, Asn and Asp at the third position; an equimolar mixture of His, Asn and Aspat the fourth position; Leu at the fifth position; and an equimolar mixture of Gly and Phy at the sixth position. The hexapeptide pool was subjected to cell-based assay at concentrations of 1.2 mM and 120 μM in order to identify effects thereof (A in FIG. 2).

As a result of the experiment, the peptide having Gly at the sixth position from the amino terminal showed more robust melanogenesis-inhibiting activity than peptides containing Phe at the same position. Further, among peptides having Gly at the sixth position from the amino terminal, the peptide having His at the third position from the amino terminal more effectively inhibited melanogenesis, compared to other peptides having Asn or Asp at the same position. That is, the peptide mixture having an amino acid sequence of (Ile/Phe)-(Asn/Ser)-His-(His/Asn)-Leu-Gly was identified as exhibiting the highest melanogenesis-inhibiting activity.

Moreover, 8 types of individual hexapeptides having specific amino acids at respective positions in the amino acid sequence were synthesized (B in FIG. 2). Each of these hexapeptides includes: His, Leu and Gly amino acids at third, fifth and sixth positions, respectively, from the amino terminal; in addition, Ile or Phe at the first position from the amino terminal; Asn or Ser at the second position; and His or Asn at the fourth position, and was designated as B1 to B8 (B1 peptide, Ile-Asn-His-His-Leu-Gly, SEQ ID NO: 1; B2 peptide, Ile-Ser-His-His-Leu -Gly, SEQ ID NO: 2; B3 peptide, Ile-Asn-His-Asn-Leu-Gly, SEQ ID NO: 3; B4 peptide, Ile-Ser-His-Asn-Leu-Gly, SEQ ID NO: 4; B6 peptide, Phe-Asn-His-His-Leu-Gly, SEQ ID NO: 5; B6 peptide, Phe-Ser-His-His-Leu-Gly, SEQ ID NO: 6; B7 peptide, Phe-Asn-His-Asn-Leu-Gly, SEQ ID NO: 7; B8 peptide, Phe-Ser-His-Asn-Leu-Gly, SEQ ID NO: 8). The individual peptides were identified, as to melanin inhibitory effects at 100 μM and 10 μM concentrations.

All analyzed hexapeptides significantly inhibited intracellular melanogenesis at a concentration of 100 μM, whereas only one type of hexapeptide showed some melanogenesis inhibitory activity at 10 μM. Generally, Phe was more effective than Ile at the first position from the amino terminal; likewise, Ser rather than Asn at the second position; and His rather than Asn at the fourth position, were effective in inhibiting intracellular melanogenesis. Therefore, B6 peptide having an amino acid sequence of Phe-Ser-His-His-Leu-Gly was shown to be the hexapeptide having strongest melanogenesis inhibitory effect.

### EXAMPLE 3

Melanogenesis Inhibitory Effect of Short Peptide

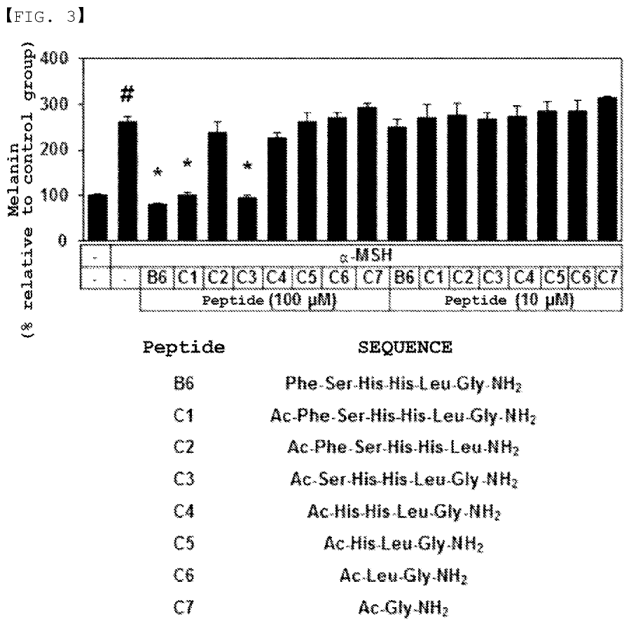

In the foregoing examples, the B6 peptide having an amino acid sequence of Phe-Ser-His-His-Leu-Gly was found to have the strongest melanogenesis inhibitory effect. Additionally, in consideration of convenience and economic advantage for peptide synthesis, peptides having shorter sequence than the hexapeptide, which are expected to be more advantageous, were examined in terms of melanogenesis inhibitory effect and availability (FIG. 3).

For this purpose, C1 to C7 peptides were further synthesized as follows: C1 (Phe-Ser-His-His-Leu-Gly; a peptide having the same amino acid sequence as B6 peptide, wherein the amino terminal and the carboxy terminal were acetylated and amidated, respectively); C2 (Phe-Ser-His-His-Leu, SEQ ID NO: 9); C3 (Ser-His-His-Leu-Gly, SEQ ID NO: 10); C4 (His-His-Leu-Gly, SEQ ID NO: 11); C5 (His-Leu-Gly, SEQ ID NO: 12); C6 (Leu-Gly, SEQ ID NO: 13); C7 (Gly, SEQ ID NO: 14). All of the C1 to C7 peptides have acetylated amino terminals and amidated carboxy terminals by a chemical synthesis method. Effects on melanogenesis at concentrations of 100 μM to 10 μM were evaluated and compared to B6 peptide.

Among the additional analyte peptides, C1 hexapeptide exhibits a melanin inhibitory effect similar to B6 peptide, thus being observed that the acetyl functional group in the amino terminal does not lose the effects of B6 peptide. Among the shorter peptides than the hexapeptide, C3 pentapeptide wherein Phe is removed from the amino terminal of B6 peptide was excellent in melanogenesis inhibitory effect at 100 μM. In contrast, C2 pentapeptide wherein Gly is removed from the carboxy terminal of B6 peptide did not substantially inhibit melanogenesis, thus suggesting that Gly at the carboxyl terminal is functionally important. On the other hand, C4 to C7 peptides shorter than the pentapeptides aid not exhibit melanin inhibitory effect.

### EXAMPLE 4

Melanogenises Inhibitory Effect of the Hexapeptide B6 in Rodent Melanoma Cells

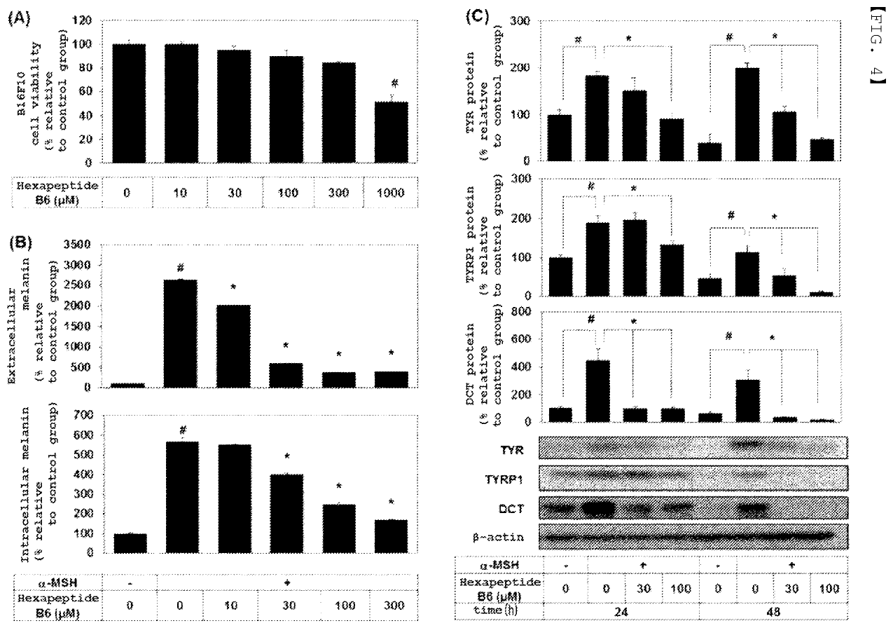

Effects of the hexapeptide B6 on cell viability and melanin content in B16-F10 cells were examined (FIG. 4).

Cell viability was determined by treating the cells with B6 at different concentrations up to 1 mM for 24 hours (A in FIG. 4). B6 peptide at up to 300 μM did not affect the viability of B16-F10 cells. Further, effects on melanin content in the cells were examined by pre-treatment of the cells with B6 peptide at different concentrations of up to 300 μM, and then, stimulating the same with α-MSH at 100 nM for 72 hours (B in FIG. 4).

In the cells stimulated with α-MSH, intracellular and extracellular melanin contents were increased and the effect of α-MSH was concentration-dependently decreased by B6 peptide. Further, the impact on expression levels of the enzyme protein, tyrosinase (TYR), tyrosinase-related protein 1 (TYRP1) and dopachrome tautomerase (DCT) was analyzed by Western blot (C in FIG. 4). In the cells stimulated with α-MSH, TYR, TYRP1 and DCT protein levels were increased 24 hours and 48 hours after stimulation. However, the cells treated with B6 peptide showed significantly decreased effects of stimulation by α-MSH.

### EXAMPLE 5

Melanogenises Inhibitory Effect of the Hexapeptide B6 in Human Epidermal Melanocytes

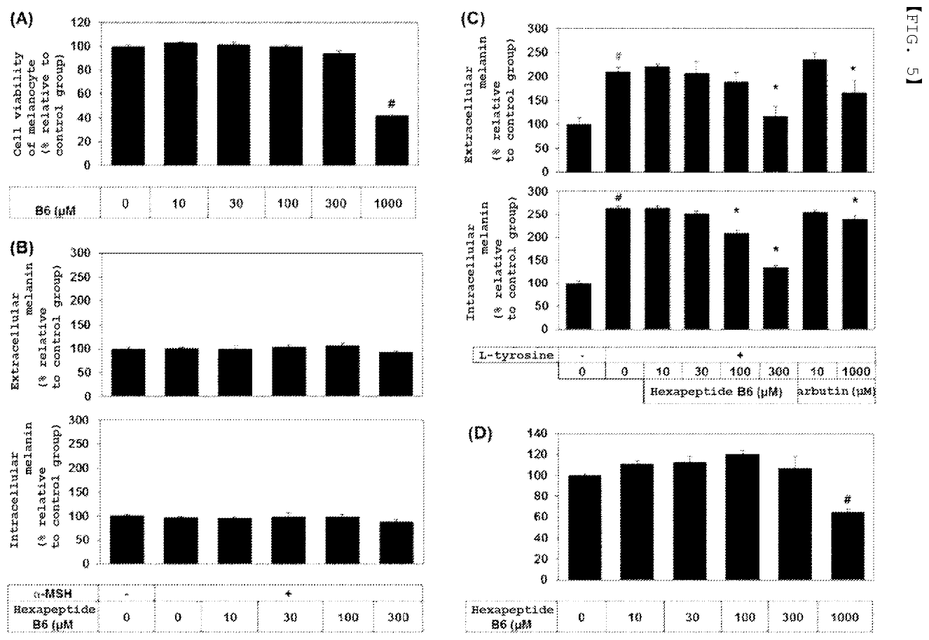

Effects of the hexapeptide B6 on cell viability and melanin content in HEM were examined (FIG. 5).

Cell viability was determined by treating the cells with B6 at different concentrations of up to 1 mM for 24 hours (A in FIG. 5). B6 peptide at up to 300 μM did not affect the viability of HEM cells. Further, effects on melanin content in the cells were examined by pre-treatment of the cells with B6 peptide at different concentrations of up to 300 μM, and then, stimulating the same with α-MSH at 100 nM or tyrosine at 4 mM for 6 hours (B, C in FIG. 5). As a positive control group for melanogenesis inhibition, arbutin was used.

Unlike B16-F10 cells, α-MSH did not show significant effect on melanogenesis. However, L-tyrosine increased extracellular and intracellular melanin contents of HEM cells. An increase in melanin content by L-tyrosine was effectively inhibited by B6 and arbutin. In fact, B6 peptide was much more effective than arbutin. Arbutin is a functional component used in whitening cosmetics, but B6 peptide having much higher effects than arbutin may be very useful as a whitening material. Moreover, it has been seen that B6 peptide at 1 mM may inhibit enzymatic activity of TYR-TYR in HEK293 cells (D in FIG.5). The HEK293-TYR cell is a transformed cell line that does not express other melanin biosynthesis-related enzymes except for human TYR. These results show that the B6 peptides effectively inhibit melanogenesis in human melanocyte cells.

### Advantageous Effects

The peptide having melanogenesis-inhibiting activity according to the present invention can be usefully applied to development of safer and more effective medicines, cosmetics and/or functional foods for whitening.

1. A peptide having melanogenesis-inhibiting activity, comprising an amino acid sequence represented by general formula (I) below,
(I) an amino terminal-X1-X2-X3-Leu-X4-carboxy terminal;
wherein,
X1 is Asn or Ser;
X2 is any one amino acid selected from group consisting of Asn, His and Asp;
X3 is His or Asn; and
X4 is Gly or Phe.

2. The peptide according to claim 1, wherein the amino acid sequence further includes Ile or Phe at the amino terminal.

3. The peptide according to claim 1, wherein the X2 is His.

4. The peptide according to claim 1, wherein the X4 is Gly.

5. The peptide according to claim 1, wherein the peptide includes an amino acid sequence represented by SEQ ID NO. 10.

6. The peptide according to claim 2, wherein the peptide includes amino acid sequences represented by SEQ ID NO. 1 to SEQ ID NO. 8.

7. A pharmaceutical composition for whitening, including the peptide according to claim 1 as an active ingredient.

8. A cosmetic composition for whitening, including the peptide according to claim 1 as an active ingredient.

9. A food composition for whitening, including the peptide according to claim 1 as an active ingredient.

10. A method of inhibiting melanogenesis, comprising administering the peptide according to claim 1 to any individual subject.
